(19) United States
(12) Patent Application Publication
(10) Pub. No.: US 2010/0030092 A1
(43) Pub. Date: Feb. 4, 2010

(54) Adaptive Hypoglycaemia Alert System and Method

(75) Inventors: Leif Engmann Kristensen, Frederiksberg (DK); Tue Deleuran, Bagsvaerd (DK); Henrik Bengtsson, Taastrup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 12/447,067

(22) PCT Filed: Nov. 14, 2007
(86) PCT No.: PCT/EP2007/062341

§ 371 Date: Oct. 7, 2009

(30) Foreign Application Priority Data
Nov. 14, 2006 (EP) 06023605.6

(51) Int. Cl. A61B 5/0402

(52) U.S. Cl. 600/509

(57) ABSTRACT

The onset or presence of a hypoglycaemic condition in a living being is estimated by obtaining ECG signals, which are processed together with secondary input, such as skin impedance, information about a condition of the living being, or blood glucose level, to estimate whether the living being is experiencing or approaching a hypoglycaemic condition. An output alert may be generated. Feedback indicative of a possible misestimate of the onset or presence of the hypoglycaemic condition is received to allow a system incorporating the invention to adapt and personalize to a specific living being. The feedback may influence one or more sensitivity or specificity settings. Settings of the system may be influenced by the location or rate of movement of the living being.

### TECHNICAL FIELD

The present invention relates generally to systems for detecting the presence or onset of a hypoglycaemic condition in a living being, for example in a diabetes patient. The systems of the present invention may be provided as portable devices and may for example be included in a medication delivery apparatus.

### BACKGROUND OF THE INVENTION

The phenomenon of hypoglycaemia is known to be critical, in particular in living beings suffering from insulin dependant diabetes. Hypoglycemia is a physiological condition where the living being suffers from a low level of blood glucose, usually described as a condition, in which the blood glucose level of the living being decreases below a certain value. Blood glucose levels below approx. 2.5 mmol/L may give rise to serious symptoms and may potentially even become fatal to a diabetic patient, in particular, if the patient does not become aware of the condition, e.g. because the patient is asleep or preoccupied with an activity, e.g. driving a car.

As is known in the art, a glucose crash occurs when blood glucose levels of an individual are in a state of rapid decline and its symptoms are similar to hypoglycaemia. The symptoms are caused by the dynamics of a declining glucose level and not by an absolute glucose level.

Already during the onset of hypoglycemia more moderate drops of the blood glucose level, e.g. below approximately 3.8 mmol/L, may cause epinephrine, growth hormone, and cortisol to be released, resulting in symptoms such as rise in heart rate, lowering of the heart rate variability and increased perspiration, and others.

Both due to the risks of reaching unhealthy or even fatal physiological conditions, and due to the desire to be able to normalize the glycemic level in the body, there is a need to be able to monitor the physiological conditions of a living being on a continuous basis.

Invasive procedures for hypoglycaemia detection are well known, i.e. procedures including sampling of portions of interstitial fluid from the living being and measuring the level of blood glucose in the sampled portions. Different non-invasive sensors have also been proposed for obtaining a measure of blood glucose constituents in blood, e.g. using infrared BGM.

An alternative approach to detection of an absolute level of glucose plasma in the body of a living being is monitoring the state of different characteristic physiological parameters which correspond to one or more specific physiological conditions of the patient. For example, a patient being at the onset or experiencing a hypoglycaemic condition shows various different symptoms such as a change in skin impedance arising from increased perspiration, reduced body temperature as well as alterations in the ECG, i.e. T-wave is flattened, QT interval is prolonged, the heart rate increases as well as a lowering of the variability of the heart rate, etc.

Such symptoms need, however, not be caused by an approaching or occurring hypoglycaemic condition. For example, even moderate physical exercise may result in several of the above symptoms, for which reason the occurrence of some or all of the above symptoms do not necessarily indicate critical onset or presence of a hypoglycaemic condition. However, if some of the symptoms occur while the living being is asleep or driving a car, rather than when the living being is performing physical exercise, the symptoms may well indicate the onset or presence of a critical hypoglycaemic condition

### SUMMARY OF THE INVENTION

It is an object of preferred embodiments of the present invention to provide a system capable of estimating the onset or presence of a hypoglycaemic condition in a living being, which is at least partially based on ECG signals, and which is capable of estimating the onset or presence of a hypoglycaemic condition with increased accuracy.

The present invention provides a system for estimating the onset or presence of a hypoglycaemic condition in a living being, the system comprising a memory, a processor, and:
at least one first sensor element for obtaining ECG signals representative of the living being's ECG;
at least one secondary input element for receiving at least one secondary input parameter;
an output generator for generating an alert signal;
   the processor being programmed to:
process the ECG signals, the at least one secondary input parameter and at least one reference value stored in the memory to estimate the onset or presence of the hypoglycaemic condition;
cause the output generator to generate the alert signal in case the hypoglycaemic condition is occurring or approaching;
receive a feedback input indicative of a possible misestimate of the onset or presence of the hypoglycaemic condition, adjust said reference value in response to the feedback input, and store the adjusted reference value in the memory.

The invention also provides a method of generating an alert signal in case of detection of the onset or presence of a hypoglycaemic condition in a living being, comprising:
obtaining ECG signals representative of the living being's ECG;
receiving at least one secondary input parameter;
processing the ECG signals, the at least one secondary input parameter and at least one reference value to estimate the onset or presence of the hypoglycaemic condition;
generating an alert signal if the onset or presence of the hypoglycaemic condition is estimated;
receiving a feedback input indicative of a possible inaccuracy of the estimation of the detecting of the onset or presence of the hypoglycaemic condition;
adjusting the reference values in response to the feedback input;
storing the adjusted reference value.

It will be appreciated that the provision of a feedback input, in response to which the at least one reference value may be adjusted, results in an adaptive system, which is capable of adapting itself to the physiology of a particular living being, i.e. a personalised adaptive system. The present inventors have realised that default settings do not fit all living beings, and that measures have to be taken to adapt a hypoglycaemia warning system to individual living beings. The present system is capable of learning by taking a feedback into account and adjusting reference values for the hypoglycaemia detection in response to the feedback.

The reference value may include one single value or multiple values, such as threshold values for signals or parameters, sensitivity and/or specificity settings etc, as elaborated further below.

The ECG signals may be obtained by equipment known per se. For example, delivery patches incorporating ECG electrodes and ECG preamplifiers are known in the medical art for the sensing of therapeutical electrical outputs from implantable devices like pacemakers or implantable cardioverter defibrillators (ICD's). As indicated, these patches rely on an implanted device which provokes a therapeutical output. Such systems are known from, e.g., WO 02/87681. The sensed ECG signals which are recorded or transmitted may contain information regarding parameters such as heart rate, heart rate variability (HRV), QT interval, QT dispersion, polarity of T-wave, amplitude of T-wave, T-wave ratio, QRS amplitude, QRS length etc. One or more of the parameters mentioned above may be measured. Also, the respiration rate may be extracted from the recorded ECG signals.

The secondary input parameter may comprise a detected or measured value, a parameter communicated to the system via appropriate communication means, and/or a value entered as a user input. For example, the secondary input parameter may comprise at least one of the living being's skin impedance, galvanic skin resistance, skin impedance spectra, a radio-spectroscopy signal, the living being's body or skin temperature, an acoustic signal, such as acoustic sounds of the heart picked up by a microphone, a signal indicative of optical reflection of the patient's skin, a signal indicative of respiration characteristics of the living being, such as respiration rate, the living being's pulse (heart rate), the living being's blood glucose level, measured by Blood Glucose Monitoring (BGM) or Continuous Glucose Monitoring (CGM), or a manually entered user-input, electromyograms (EMG), ambient temperature, oxygen saturation, blood pressure, lung wetness, thoracic impedance, ICV pressure, CO₂ content of blood, EEG, mean or peak frequency of the alpha wave, position of patient, movement of body, pH-value.

Embodiments of the invention comprising or utilizing a blood glucose meter may be capable of communicating with the processor via a wired or wireless communication interface.

In case of a manually entered user-input, such input may be indicative of at least one of a personal condition, a metabolic state or parameter, and the living being's physical activity. The input indicative of the personal condition may indicate a physical condition, such as the living being's level of fatigue, or a psychological condition, such as a level of exciteness or stress, or an input indicative of an observed symptom of a hypoglycaemic episode. The metabolic state or parameter may be a variable or permanently set value indicative of the living being's actual and/or average metabolic balance. Such information may contribute to the prediction of a future hypoglycaemic condition, as it may contribute to the determination of a rate of decrease of the living being's blood glucose level. The input indicative of the living being's physical activity may e.g. include input regarding whether the living being is at rest, is performing moderate physical activity or intense physical activity, is at sleep, etc. Such information may also contribute to the determination of a rate of decrease of the living being's blood glucose level and thereby to the prediction of a future hypoglycaemic condition.

In preferred embodiments, the onset or presence of the hypoglycaemic condition is estimated without receiving an EEG signal as an input. Thereby, the need for EEG sensors, which often need to be placed by surgical intervention, and processing of EEG signals is eliminated.

All signals and parameters used in the estimation of the onset of occurrence of a hypoglycaemic condition, except signals and parameters used in the determination of the living being's blood glucose level, are preferably achieved in a non-invasive manner. Thus, in embodiments, which do not include BGM or CGM, the operator of the apparatus is alleviated of the burden of performing invasive measurements. It is even contemplated that non-invasive procedures may be applied for obtaining a measure of blood glucose constituents in blood, e.g. infrared BFM. The system of U.S. Pat. No. 5,741,211 for providing an indication of either blood insulin or blood glucose may also be applied. In embodiments of the present system and method, in which invasive measures are applied for obtaining an indication of the living being's blood glucose level, all other signals and parameters are preferably obtained in a non-invasive manner to minimise the inconveniences of invasive procedures.

The alert signal may be selected from the group consisting of an audible signal, a visual signal, a tactile signal, an electro-muscle stimulation, a vibratory signal, and any combination of these. Alternatively, the alert signal may include a communication signal, upon which an external device may alert of the estimated onset or occurrence of the hypoglycaemic condition.

The reference value for the ECG input and the at least one secondary input parameter may be a fixed or variable value. It may be a threshold value for specific ECG and secondary characteristics, e.g. a threshold value for the living being's heart rate, a time lapse between past medication, in particular insulin medication, dose of past medication, and/or blood glucose level. In addition, or alternatively, reference values may exist for first or second order derivates of the ECG and secondary characteristics, so that a sudden decrease in e.g. blood glucose level may result in the generation of an alert signal. A plurality of reference values may exist, e.g. one for each of several ECG characteristics, and one for secondary characteristics. The at least one reference value may be a value, which varies with one or more conditions or inputs. In one simple example, the reference value may be set for a first order derivate of blood glucose level in such a way that it is influenced by the living being's physical activity as a secondary input parameter. Thus, for example, a relatively low first order derivative of the blood glucose level is accepted if the living being is asleep, whereas a higher first order derivate may be accepted if the living being is performing moderate physical activity. In more complex embodiments, set points for the at least one reference value is determined based on more parameters and/or possibly derivatives thereof.

It will hence be understood that the at least one reference value may comprise at least one sensitivity setting. The sensitivity setting may include a value or parameter, which determines or controls the sensitivity of the processing, i.e. influences the at least one reference value. The sensitivity setting may be one, which is set by an operator of the apparatus, or it may be one, which comes from the feedback input, i.e. the feedback input may comprise at least one sensitivity-influencing input, such as a manual or automatic input indicative of a possible misestimate of an alert, or, in the alternative, an input confirming an alert as correct. The sensitivity setting may be varied under influence of the sensitivity-influencing input.

In the present context, the term “sensitivity setting” may be understood as a setting which influences or controls the system's sensitivity to the ECG signals and/or signals provided by the secondary input element. The sensitivity setting may be expressed as a threshold value for a given parameter or signal or a derivative thereof, or it may be expressed in terms of a period of time, during which the signal or parameter in question may be allowed to exceed a predetermined value. Hence, a signal or parameter of an ECG measurement, which is indicative of the onset of a hypoglycaemic condition may be allowed to exist for a period of time, before the alert signal is generated. If the signal or parameter, within that period of time, drops back to a state or value which is not indicative of the onset of a hypoglycaemic condition, no alert signal is provided, whereas if the signal or parameter stays beyond a threshold for the said period of time, the alert signal is generated. That period of time, which is allowed to lapse before an alert signal is generated, may e.g. be defined by (or be defines as) a sensitivity setting.

Likewise, a specificity of the present system may be set. Specificity may be regarded as a characteristic of the frequency of misestimates. Hence, a specificity of 100% indicates no misestimates, whereas a specificity of 90% indicates 10% misestimates. Setting a relatively high sensitivity will accordingly result in relatively many misestimates, i.e. a relatively low specificity and vice versa. The processor of the system of the present invention may be programmed to adjust itself in case of a relatively low specificity. Such adjustment may e.g. be based on statistical processing of operator feedback indicative of the occurrences of misestimates, whereby the processor is programmed to redefine or change the at least one reference value and/or the sensitivity in case of the occurrence of a too high number of misestimates. A too low number of misestimates may also result in a change in the at least one reference value and/or the sensitivity, as few misestimates indicates that safety might be compromised.

The sensitivity-influencing input may comprise at least one of: quantity and/or dose of a past medication of the living being, time lapse since a past medication of the living being, the living being's blood glucose level, e.g. past and/or current blood glucose level, input describing the living being's past meal and/or drink intake, time, a physical location of the living being, an input, upon which the level of the living being's physical activity can be determined, and/or any a manually entered input. For example, the sensitivity may depend on the time of the day or information about recent food or drink intakes. In one embodiment, the processor is programmed to set a certain sensitivity in case the physical location of the living being is a vehicle, an office, a bed room, a beach, swimming or pool area, a garden, a garage etc. Normally, a relatively high sensitivity is set in case the physical location is any other location, at which the occurrence of a hypoglycaemic condition would imply the risk of fatal consequences. However, other scenarios are possible. For example, one person may wish to set a relatively high sensitivity, when he is asleep, so as to assure that he is woken up in case of the onset or presence of a hypoglycaemic condition. Another person may, on the contrary, wish to set a relatively high specificity, and hence a relatively low sensitivity, so as to assure that he is only woken up in case of a serious risk of hypoglycaemia. The location and/or physical activity of the living being may also be determined automatically. For example, the system may include an accelerometer and/or GPS features allowing it to determine e.g. the rate of movement or acceleration of the living being.

The sensitivity-influencing input may comprise an input, upon which the level of the living being's physical activity can be determined, in which case the processor may be programmed to set a certain sensitivity depending on the level of the living being's physical activity, for example a relatively high sensitivity in case the living being's physical activity is high.

The system may comprise a user-interface allowing a user to directly set the sensitivity. For example, a number of sensitivity levels may be pre-programmed into the system, in which case a user may select that sensitivity level, which serves him best in a given situation. Thereby, the user is given maximum control over the system. In other embodiments, the sensitivity is set or determined automatically based on various inputs, as described herein.

To detect the physical location of the living being, the system of the present invention may further comprise a location detector. For example, in embodiments in which at least the processor and the output generator are incorporated in a portable device, such as a skin-borne device, and wherein the location detector is a separate, external element adapted to be secured to a specific physical location, the system may further comprise a location interface causing the system to be set at a certain sensitivity, e.g. a relatively high or a relatively low sensitivity, when the location detector is within communication reach of the portable device. Such communication reach may either be through a wireless or wired communication interface. The sensitivities may be set or determined automatically by the system, or they may be provided as a user input.

An operator of the system of the present invention may be allowed to set a detection or sensitivity mode of the system. For example, one mode may be “garden work”, whereas another mode may be “driving a car”. Other modes may be “office work”, “low-”, “medium-” or “high-impact exercise”, or “light-”, “medium-”, or “large snack”, “extended meal” etc. The memory of the system may include a plurality of predefined modes, which may be selected by the operator. Each mode defines or influences a sensitivity and/or specificity of the system, and/or the at least one reference value for the ECG signal and/or the secondary input parameter.

At least the processor and the output generator may be incorporated in a portable device, such as a skin-borne device. The portable device may comprise a medication delivery apparatus housed within the portable device, such as a delivery device for insulin.

### DESCRIPTION OF THE DRAWINGS

Embodiments of the invention will now be further described with reference to the drawings, in which:

FIGS. 1-5 are block diagrams illustrating a system according to the present invention;

FIG. 6 is a simplified flowchart illustrating a basic mode of operation of the present invention;

FIG. 7 shows two examples of different sensitivity modes.

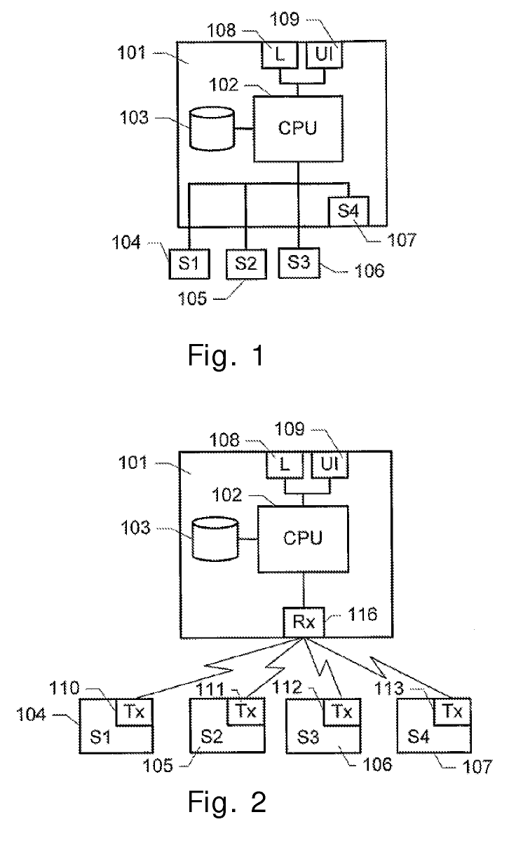

FIGS. 1-5 show block diagrams of examples of an apparatus for detecting hypoglycaemia. In the following, same reference numbers refer to the same components.

Referring to FIG. 1, the system 101 comprises a processor 102, a memory 103, a loudspeaker 108, and a user interface 109. The apparatus further comprises or is connected to a number of sensors generally designated 104, 105, 106, and 107. In the example of FIG. 1, the system 101 is connected to three sensors 104, 105, and 106 via cables, and the system 101 further comprises an integrated sensor 107, e.g. a pulse sensor or a skin temperature sensor integrated into a device, e.g. a skin-borne device, or a device which is worn around the user's wrist.

For example, the sensors 104, 105, 106, and 107 may measure the pulse, the heart rate variability, the skin temperature, and the skin impedance, respectively. However it is understood that alternative or additional measurements may be performed.

The pulse sensor may be based on any suitable method known in the art such as photoelectric measurements, e.g. as described in “The Biomedical Engineering Handbook, CRC Press, Volume 1 (ISBN: 0-8493-0461-X), p. 86-1-86-7. For example, the pulse sensor may include a pulse oximeter, e.g. placed at the user's fingertip or ear lobe.

The skin impedance may be based on any suitable method known in the art. For example, the skin impedance sensor may comprise a concentric type electrode with an outer passive electrode and an inner electrode, e.g. as disclosed in WO 02/069798.

The measurement of the hart rate variability (HRV) may be based on any suitable method known in the art, e.g. as described in “The Biomedical Engineering Handbook, CRC Press, Volume 1 (ISBN: 0-8493-0461-X), p. 13-1-13-8. For example, the HRV may be determined based on an ECG, e.g. measured via electrodes placed on the user's chest and/or arms.

The skin temperature may be measured based on any suitable method known in the art, e.g. by means of a thermistor-based sensor.

It is understood that in alternative embodiments, a different set of sensor signals may be used. In addition or alternatively to the above sensor signals such a set of sensor signals may include respiration frequency, respiration effort, eye movements, EOG, muscle tonus, parameters determined by an ECG, e.g. QT interval, frequency of the a wave, etc., parameters determined by Electroencephalography (EEG), etc., third degree sensor signals such as the O₂ and/or CO₂ content of the blood, first degree sensor signals such as a non-invasive blood glucose measurement, etc., or any combination of the above. The above parameters may be detected by any suitable method known per se in the art.

The sensors 104, 105, 106, and 107 forward the measured sensor signals to the processor 102. In one embodiment, the signals are forwarded as analogue signals which are processed by the processor, e.g. by sampling/digitizing the analogue signal and/or averaging the signals over a predetermined time, or the like. In another embodiment, some or all of the sensors 104, 105, 106, and 107 perform some or all of the above processing and forward a suitably sampled, averaged and digitized signal to the processor 102.

The processor 102 processes some or all ECG signals, secondary input parameters, as well as at least one reference value stored in the memory 103, and determines whether or not an alert should be generated.

If an alert is generated, the processing unit activates the loudspeaker 108. It is understood that alternatively or additionally, any other suitable output device for generating an alert may be used.

The system further comprises a user interface 109, including e.g. one or more push buttons, a keypad, a touch screen, a voice-recognition system, or the like, allowing the user to provide feedback to the apparatus. For example, the user interface may allow a user or operator to indicate whether or not one or more past alerts were misestimates or correct estimates of the onset or occurrence of a hypoglycaemic condition. This feedback may be used in order to adjust the reference value stored in the memory. The user interface may further allow a user to enter a secondary input parameter, e.g. a measured blood glucose level, thereby providing a feedback or secondary input about the degree of hypoglycaemia, if any.

FIG. 2 shows a further embodiment of a system according to the invention. In this embodiment, the system 101 receives the sensor signals from the sensors 104, 105, 106, and 107 via radio communication. Consequently, the apparatus 101 further comprises a short-range radio receiver 116, e.g. a receiver adapted to receive radio signals in an unlicensed radio frequency band. In one embodiment, the receiver is implemented according to the Bluetooth standard. Similarly the sensors 104, 105, 106, and 107 each comprise a corresponding radio transmitter 110, 111, 112, and 113, respectively, adapted to communicate with the receiver 116.

For example, the system 101 with the receiver, the processor 102 and the alarm output may be a device that may be placed on a night stand or it may be a watch-like unit worn around the user's wrist. In case of the nightstand device, the device may optionally include a refrigerators compartment that is sufficiently large to hold some juice, a soft drink, or the like, thereby allowing the patient to immediately counterbalance a condition of hypoglycaemia in case of an alert.

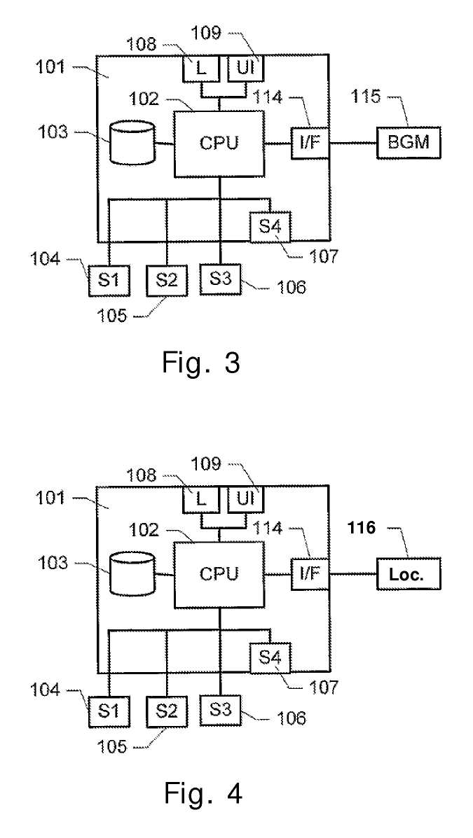

FIG. 3 shows yet another embodiment of the system of the present invention. In this embodiment, the apparatus 101 is connected to three sensors 104, 105, and 106 via cables, and the apparatus 101 further comprises an integrated sensor 107, as in the example of FIG. 1.

Additionally, the apparatus of FIG. 3 further comprises an interface circuit 114 for receiving a signal from a blood glucose measurement device 115. For example, the interface circuit may be a wired connection, a plug-and-socket connection or a wireless connection, e.g. an infrared or radio-based connection. The interface circuit 114 allows a user to directly transfer a blood glucose value measured by the measurement device 115 to the apparatus 101, thereby allowing the user to verify or reject an alarm raised by the apparatus 101.

FIG. 4 shows a similar device, in which a location detector 116 is provided for allowing the processor 102 to detect if the living being is within communication reach of the location detector 116, and hence at increased risk of hypoglycaemia.

It is understood that a number of equivalent embodiments of an apparatus may be designed, including combinations of the above examples.

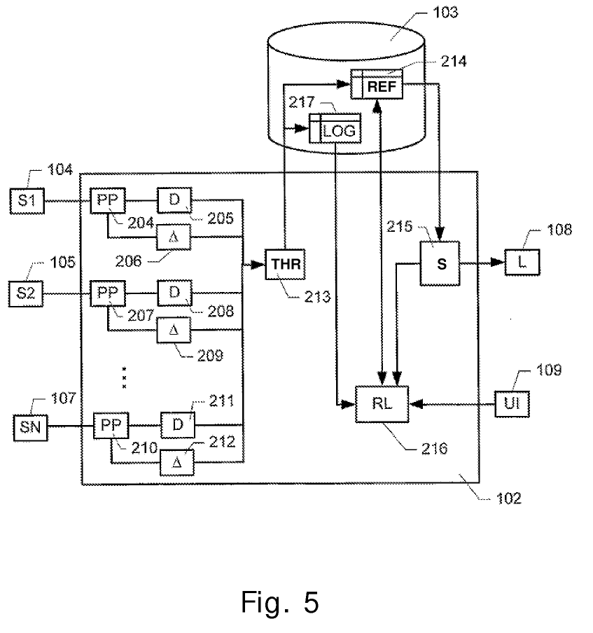

FIG. 5 shows a more detailed block diagram of the functions performed by the processor of the system according to the invention.

The processor 200 receives inputs from N sensors exemplified by sensors 104, 105, and 107, generally designated S1, S2, . . . , SN.

The signal received from sensor S1 is fed into a pre-processor module 204 where it is suitably pre-processed, e.g. averaged over a predetermined time period, e.g. a few seconds, and/or normalised and/or the like. The preprocessed signal is fed into a discretizer module 205. The discretizer module 205 determines in which of a number of predetermined intervals the received sensor signal falls. Assuming that the total range of the sensor signal S1 lies between S₁ₘᵢₙ and S₁ₘₐₓ, the range is divided into K1 intervals I_{1,1}=(S1ₘᵢₙ; S1₁], i_{1,2}=(S1₁; S1₂], . . . , i_{1,K1}=(S1_{K1-1}; S1ₘₐₓ), and the discretizer module determines the intervales, k=(S1ₖ₁;S1ₖ], such that S1ₖ₁<S1<S1ₖ.

For example, skin temperature T may be discretized as “relatively low” (corresponding to T<23 deg. C.), “low” (23 deg. C<T<25 deg. C.), “normal” (25 deg. C<T<27 deg. C.), and “high” (T>27 deg. C.). Hence, the normal value (26 deg. C.) is in the third interval, and the intervals are shifted towards the temperature range which is relevant for the detection of the onset or occurrence of hypoglycaemia, i.e. towards the temperatures below the normal value. The discretizer outputs the number of the identified interval.

The pre-processed signal is also fed into a module 206 for determining a rate of change of the sensor signal S1. Similarly to the discretization of the actual sensor signal, the rate of change is also determined as falling within one of a number of intervals. In one embodiment, the range of change may simply be determined as a difference of two consecutive values of the sensor signal.

For example, in the above example of skin temperature, the rate of change may be discretized into “rapidly decreasing”, “slowly decreasing”, “slowly increasing”, or “rapidly increasing”. The module 206 outputs an indication of the rate of change, e.g. by outputting the number of the corresponding interval.

Similarly, the sensor signal received from sensor S2 is pre-processed in preprocessor 207, discretised in discretizer module 208, and a rate of change is determined in module 209. The sensor signal received from sensor SN is pre-processed in pre-processor 210, discretized in discretizer module 211, and a rate of change is determined in module 212. Hence, in this embodiment, 2N interval numbers are generated.

The index numbers determined from the received sensor signals and the corresponding rates of changes are fed into the threshold comparison module 213. For each possible combination of the 2N intervals, the comparison module determines a corresponding reference value 214. The reference values are stored in a memory 103, e.g. an EPROM, EEPROM, a hard disk, a memory card, or the like. Each reference value corresponds to an estimated probability that the living is in a hypoglycaemic condition or close to a hypoglycaemic condition. The threshold comparison module further stores a corresponding timestamp in a log table 217 stored in the memory 103 or, alternatively, in a separate memory.

For example, the log table may store the index numbers selected during the past 12 hours, the last 24 hours, or the like.

The determined probability is fed into the sensitivity module 215, which decides if an alert signal is output by the loudspeaker 108 to sound an audible alert.

The sensitivity module further forwards a signal to a reinforcement module 216 indicating that an alarm has been triggered.

In one embodiment, the above process is repeated in regular time intervals, e.g. every 30 seconds, every minute, every few minutes, or the like. In some embodiments, an alert is only raised, if the determined probability is determined to be above threshold in a predetermined number of consecutive time intervals.

If the processor receives a signal from the user interface relating to the actual hypoglycaemic or non-hypoglycaemic condition of the user, as the case may be, the received information is fed into a reinforcement learning module 216. Based on the received input and any possible signals received from the threshold unit about any triggered alerts, the reinforcement module determines a time period such that the at least one reference value, or other settings, such as specificity or sensitivity settings, selected or set during that time period are modified. For example, if an alert has been generated by the apparatus and if the user has indicated via the user interface 109 that the alert was a misestimate, the reinforcement module may determine to decrease all probabilities that were selected during the last 30 minutes prior to the alert. Likewise, if an alert has been generated by the apparatus and if the user has acknowledged via the user interface 109 that he/she actually experiences a condition of hypoglycaemia, e.g. based on a blood glucose measurement, the reinforcement module may determined to increase all probabilities that were selected during the last 30 minutes prior to the alert. Similarly, if the received user input indicates that the user has had an undetected condition of hypoglycaemia during the previous night, the reinforcement module may determine to modify the at least one reference value or other settings that were set during the previous night. Consequently, the reinforcement module retrieves information from the memory 217 identifying the entries that were selected during the determined period of time and the corresponding points in time at which the entries were selected. The reinforcement module 216 then calculates modified probabilities for the identified entries and stores recalculated sensitivities, specificities and/or reference values.

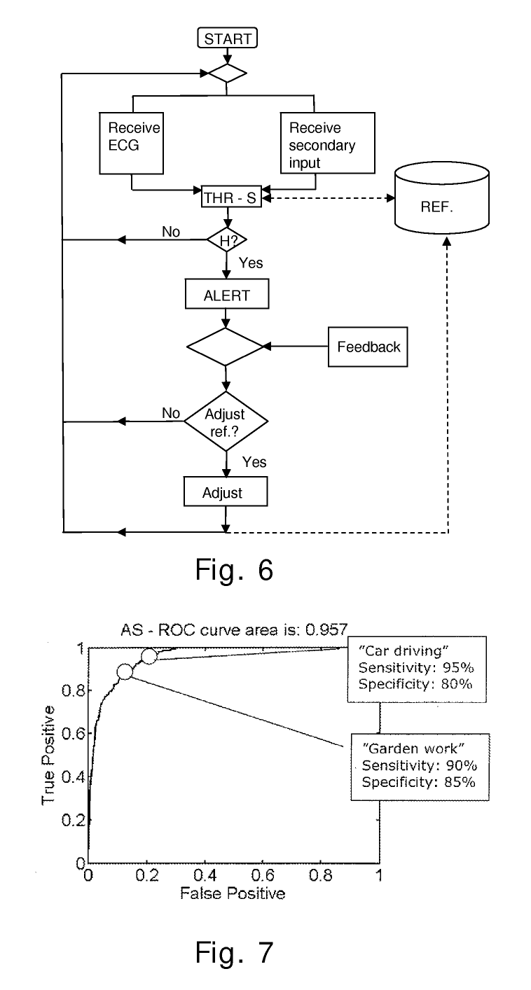

It is understood, that the functions performed by the processing unit and described with reference to FIG. 5 above may be implemented fully or partly in software, where the blocks in FIG. 5 represent different functional components. FIG. 6 illustrates a simple mode of operation of the method and system of the present invention. ECG signals and secondary input or inputs are received and processed at THR-S. To estimate the onset or occurrence of hypoglycaemia, the ECG signals and secondary input signals are processed together with one or more reference values, sensitivity settings or specificity setting, stored in database REF. Based on such processing, it is estimated whether or not a hypoglycaemic condition is approaching or occurring. If so, an alert is generated.

Feedback is received via a suitable user interface. For example, reference values, sensitivity settings, detection modes and/or specificity may be received. In case the feedback necessitates the adjustment of one or more reference values, sensitivity or specificity settings, or detection modes, such changes are stored in the memory REF.

It should be understood that user feedback may also be received in case the method estimates no presence or onset of hypoglycaemia.

FIG. 7 illustrates two different sensitivity and specificity modes, “car driving” and “garden work”. As shown, the relatively high sensitivity of the “car driving” mode, 95%, results in a relatively low specificity of 80%, whereas the relatively low sensitivity of the “garden work” mode, 90%, results in a relatively high specificity.

1. A system for estimating the onset or presence of a hypoglycaemic condition in a living being, the system comprising a memory, a processor, and:
at least one first sensor element for obtaining ECG signals representative of the living being's ECG;
at least one secondary input element for receiving at least one secondary input parameter;
an output generator for generating an alert signal;
the processor being programmed to:
process the ECG signals, the at least one secondary input parameter and at least one reference value stored in the a memory to estimate the onset or presence of the hypoglycaemic condition;
cause the output generator to generate the alert signal in case the hypoglycaemic condition is occurring or approaching;
receive a feedback input indicative of a possible misestimate of the onset or presence of the hypoglycaemic condition, adjust said reference value in response to the feedback input, and store the adjusted reference value in the memory.

2. A system according claim 1, wherein:
the at least one reference value comprises at least one sensitivity setting;
the feedback input comprises at least one sensitivity-influencing input;
the processor is programmed to vary the sensitivity setting under influence of the sensitivity-influencing input.

3. A system according to claim 2, wherein the sensitivity-influencing input comprises at least one of:
quantity of a past medication of the living being;
time lapse since a past medication of the living being;
the living being's blood glucose level;
input describing the living being's past meal and/or drink intake;
time;
dose of last medication of the living being;
a physical location of the living being;
an input, upon which the level of the living being's physical activity can be determined;
a manually entered input; and
a user-specified sensitivity setting.

4. A system according to claim 3, wherein the sensitivity-influencing input comprises the physical location of the living being, and wherein the processor is programmed to vary the sensitivity setting in case said physical location is a vehicle.

5. A system according to claim 3, wherein the sensitivity-influencing input comprises the physical location of the living being, the system further comprising a location detector for detecting said physical location.

6. A system according to claim 5, wherein at least the processor and the output generator are incorporated in a portable device, and wherein the location detector is adapted to be secured to a specific physical location, the system further comprising a location interface causing the system to change the sensitivity when the location detector is within communication reach of the portable device.

7. A system according to claim 3, wherein the sensitivity-influencing input comprises the input, upon which the level of the living being's physical activity can be determined, and wherein the processor is programmed to set change the sensitivity in response to said input.

8. A system according to claim 1, further comprising an operator interface for selecting a detection mode, and wherein said sensitivity setting is influenced by the selected detection mode.

9. A system according to claim 1, wherein said at least one secondary input parameter comprises at least one of:
the living being's skin impedance;
a radio-spectroscopy signal;
the living being's body temperature;
an acoustic signal;
a signal indicative of optical reflection of the patient's skin;
a signal indicative of respiration characteristics of the living being;
the living being's pulse;
the living being's blood glucose level;
a manually entered user-input;
time;
input describing the living being's past meal and/or drink intake; and
a signal indicating the living being's activity.

10. A system according to claim 9, wherein the manually entered user-input is indicative of at least one of:
a personal condition;
a metabolic state or parameter;
the living being's physical activity; and
the living being's location.

11. A system according to claim 1, wherein the processor is programmed to estimate the onset or presence of the hypoglycaemic condition without receiving an EEG signal as an input.

12. A system according to claim 1, wherein at least the processor and the output generator are incorporated in a portable device.

13. A system according to claim 12, further comprising a medication delivery apparatus housed within the portable device.

14. A system according to claim 1, wherein said at least one secondary input parameter comprises the living being's blood glucose level measured by Blood Glucose Monitoring (BGM) or Continuous Glucose Monitoring (CGM).

15. A system according to claim 14, comprising a blood glucose meter capable of communicating with the processor via a wired or wireless communication interface.

16. A system according to claim 1, wherein said at least one secondary input parameter comprises a manually entered user-input indicative of an observed symptom of a hypoglycaemic episode.

17. A method of generating an alert signal in case of detection of the onset or presence of a hypoglycaemic condition in a living being, comprising:
obtaining ECG signals representative of the living being's ECG;
receiving at least one secondary input parameter;
processing the ECG signals, the at least one secondary input parameter and at least one reference value to estimate the onset or presence of the hypoglycaemic condition;
generating an alert signal if the onset or presence of the hypoglycaemic condition is estimated;
receiving a feedback input indicative of a possible inaccuracy of the estimation of the detecting of the onset or presence of the hypoglycaemic condition;
adjusting the at least one reference value in response to the feedback input; and
storing the adjusted reference value.

18. A method according to claim 17, wherein the at least one reference value comprises at least one sensitivity setting and the feedback input comprises at least one sensitivity-influencing input.

19. A method according to claim 18, wherein the sensitivity-influencing input comprises at least one of:
quantity of a past medication of the living being;
time lapse since a past medication of the living being;
the living being's blood glucose level;
input describing the living being's past meal and/or drink intake;
time;
dose of last medication of the living being;
a physical location of the living being;
an input, upon which the level of the living being's physical activity can be determined;
a manually entered input;
a user-specified sensitivity setting.
